(19) United States
(12) Patent Application Publication
(10) Pub. No.: US 2020/0260685 A1
(43) Pub. Date: Aug. 20, 2020

(54) METHOD AND APPARATUS FOR REARING SMALL ANIMALS

(71) Applicant: Zyzzle Limited, Surbiton (GB)

(72) Inventor: Glen Slade, Surbiton (GB)

(21) Appl. No.: 16/868,191

(22) Filed: May 6, 2020

Related U.S. Application Data
(63) Continuation of application No. PCT/GB2018/053227, filed on Nov. 7, 2018.

(30) Foreign Application Priority Data
Nov. 8, 2017 (GB) 1718479.7
Sep. 28, 2018 (GB) 1815868.3

(51) Int. Cl. A01K 1/03; A01K 29/00; A01K 1/00; B25J 11/00

(52) U.S. Cl. CPC A01K 1/031; B25J 11/00; A01K 1/0047; A01K 29/005

(57) ABSTRACT

Apparatus which comprises a plurality of containers (2) for holding animals (1, 4), wherein the conditions in each container are isolated from the conditions in other containers. The apparatus is provided with a measurement device (3, 10) for taking a measurement related to a first container or an animal associated with the first container. An actuator (5, 11) is also provided to perform an action on the first container, its environment or on an animal associated with the first container. The apparatus is particularly useful for providing an improved animal population and rearing process. It is particularly useful for rearing insects.

### CROSS-REFERENCE TO RELATED APPLICATIONS

This application is a Continuation of and claims the benefit of priority under 35 USC 120 to PCT/GB2018/053227 filed Nov. 7, 2018, which claims priority to GB1718479.7 filed Nov. 8, 2017 and GB1815868.3 filed Sep. 28, 2018, the entire contents of each are hereby incorporated by reference.

### BACKGROUND OF THE DISCLOSED SUBJECT MATTER

### Field of the Disclosed Subject Matter

The present invention provides apparatus for rearing small animals and methods of rearing small animals. More specifically the invention relates to the rearing of insects such as mosquitoes.

Conventional rearing systems for small animals typically utilize containers to hold the animals wherein each container contains a large number of animals. For example, in the production of Aedes aegypti mosquitoes, each container generally holds tens of thousands of individuals. The document WO2014/171829 A1 discloses a system for breeding insects using a number of crates.

Rearing conditions are provided to attempt to maximize the yield rate. However, due to natural variation in the animals, a significant proportion of animals is inevitably not suited to the conditions and do not survive the rearing process. The natural biological variation in a population of a particular species means that planning a rearing process on the median specimen is sub-optimal for a sub-population in each rearing container.

In some cases measurements are taken to try to tailor the rearing conditions which are applied to all of the animals. However, in this case the yield is adversely affected due to the high proportion of animals that do not thrive or survive when the conditions of the process are changed.

### SUMMARY OF THE DISCLOSED SUBJECT MATTER

It is amongst the objectives of the present invention to address one or more of the problems outlined above.

According to a first aspect of the invention there is provided an apparatus for rearing animals comprising;

a plurality of containers for holding animals, wherein each container is adapted to accommodate controlled conditions which are isolated from the conditions in the other containers, and the volume of each container is no greater than 0.001 m³,

a measuring device which is adapted to perform a measurement on a first container of the plurality of containers, the environment of the first container, an animal within the first container or an animal removed from the first container, and

an actuator arranged to perform an action on the first container, the environment of the first container, an animal in the first container or an animal removed from the first container.

The isolation of a particular condition associated with each container, coupled with the measuring device allows the isolated conditions in each container to be monitored accurately. The actuator allows the conditions in a particular container to be changed, or an action to be taken on an animal in a particular container, optionally in response to the specific conditions or the measurement taken on an animal in that particular container. The isolation of the containers, the measuring device and the actuator therefore provide a way of exposing small groups of animals to highly individualized rearing conditions. This can achieve higher yield than could be achieved with growth en masse. If uniform animal fitness is the objective, animals that are measured to be slightly larger than average may require slightly more food while those that are slightly smaller than average may require slightly less food. If the objective is to rear animals of uniform size, actions can be taken to slow the growth of larger individuals and promote the growth of smaller individuals. If the objective is to synchronize growth, for example so that a group of animals enters a life stage such as pupation at a more closely aligned time (which may in turn optimize a storage or transport window), then steps can be taken to slow the development of more developed individuals and accelerate the development of less developed individuals. When the measurement relates to a characteristic of the animal, the characteristic measured may be the animal's size, colour, weight, stage of development, whether the animal is alive, or a combination thereof.

Secondly, reflecting the potential conflict between maximizing animal fitness and achieving uniformity of growth and/or development, the present invention may be used to select, across a much wider range of developing individuals, a subset that meets a specific need. Where animals are reared in a batch process, individuals can be ‘promoted’ or ‘relegated’ between virtual batches to minimize waste and/or maximize quality in the batch. Where animals are reared in a continuous process, the same concept applies by treating each animal as a batch of one.

Thirdly, the ability to vary specific rearing parameters by individual creates significant opportunity to research optimized rearing including (without limitation) feeding and climate.

Fourthly, the measurement may provide the ability to reject an animal as soon as measurements show that it is inappropriate for the objective. This enables feeding and other resources to be stopped as soon as possible, saving costs. Rejected individuals can be removed or sanitized promptly to avoid fungal contamination or other spoiling.

Fifthly, because the measurement may be made on a small number of animals or on an individual animal, quality control is significantly more accurate than in systems based on mass sampling. The actuator may be adapted to add feed or set other conditions based on the exact number of animals rather than the estimated size of a cohort as has been the case in the prior art. The actuator may achieve high accuracy sex separation for ‘sterile insect technique’ applications. The actuator may also be adapted to prevent animals escaping or dying if they are outside the growth norms for a given point in the rearing process (for example, insects becoming adult ‘flyers’).

When it is stated that each container is adapted to accommodate controlled conditions which are isolated from the conditions in the other containers, this is intended to encompass containers in which only one particular characteristic of the conditions in each container is isolated from the other containers. For example, in some embodiments only the temperature of each container is isolated from the other containers. In other embodiments, only a volume of a growth medium in one container is isolated from a volume of growth medium in other containers. However, two or more of the conditions, for example the temperature and the amount of nutrient in one container may be isolated from the temperature and nutrients in another container.

A measurement on a first container may include a measurement of the contents of the first container. When rearing mosquitoes, there will inevitably be water, discarded skins, other debris, waste products and other contaminants in the container. It may be useful to take a measurement of the presence or quantity of any of these components to check whether their presence or quantity could detrimentally affect the animal. Appropriate action can then be taken by the actuator. For example, if a lower than desired water level is measured, the amount of water may be topped up.

An action performed by the actuator need not be made in response to a measurement obtained by the measuring device or a status monitored by the monitoring device. The actuator may also perform pre-set actions, which occur for example at predetermined time intervals. This might for example involve the addition of further food to a container at time t1, without making a specific measurement. Equally, the actuator could be arranged to add food to a container in a first batch, but not to a container in a second batch.

In some embodiments the apparatus may further comprise a monitoring device for monitoring a status, wherein the status is of the apparatus, an animal associated with the apparatus or a process associated with the apparatus.

The status of an animal may include its identifier and/or batch number or its location on a plate or tray which comprises the containers. Determining the status is useful because a user might wish to, for example, feed a first batch of animals every day and only feed a second batch on alternate days. The system status variables may include the number of empty containers in a tray (e.g. due to animal removal), in the case where the apparatus comprises a tray or several trays which comprise the plurality of containers. Monitoring this status may be useful because this information can inform the actuator whether to consolidate animals into a single tray. The process status may include the current mortality rate of a particular batch, in order to trigger an additional action or an alert to a user.

The measuring device or the monitoring device may be arranged to communicate with the actuator so that an action performed by the actuator may be made in response to a measurement obtained by the measuring device or a status monitored by the monitoring device.

In preferred embodiments the measuring device and/or the actuator and/or the monitoring device are connected to a control system, such as a computer. This may allow data from the measuring device and/or the actuator to be recorded and reviewed. It may also allow the control system to use data from the measuring device and/or monitoring device to determine whether the actuator should carry out a specific action. Based on the data received, the control device may also send an instruction to the actuator to carry out a particular action and/or to the measuring device to make a particular measurement, and/or to the monitoring device to obtain a status.

This avoids the need for a user to instruct the actuator to carry out a particular action. The apparatus may be programmed to perform specific actions if a particular measurement outcome is obtained. For example, if the animal is a mosquito larva which is measured to be smaller than the average size for animals in the process, the actuator may be programmed to add a food supplement to the container which accommodates that larva. Furthermore, the action performed by the actuator may be quantitively different for different measurement outcomes. For example the amount of food or water which is added to or removed from a container might be varied depending on the size of an animal which is measured. The apparatus may be pre-programmed accordingly.

The action can therefore address each animal or each animals' needs individually, or as a small group, based on the results of the measurement. This provides much more specific rearing conditions than methods of the prior art, which are not tailored to individual animals or to small groups of animals.

The apparatus may be arranged to provide individualized rearing conditions for each individual animal. Individualized rearing allows conditions to be provided that are better suited to each individual animal in order to achieve a higher yield rate or other user objective. Longer hatching times may be used since the animals may be placed into containers as and when they hatch. This increases the hatching rate and hence the yield.

The measurement may be of: the temperature in the first container, the humidity in the first container, the illumination of the first container, the turbidity of contents of the first container, the pH of contents of the first container, food quantity within the first container, a characteristic of food in the first container, a characteristic of an animal such as the dimensions of an animal, weight of an animal, color of an animal, health of an animal, or the sex of an animal in the first container, the cleanliness of the first container, or a combination thereof.

By measuring any of these properties the rearing conditions can be adapted to the needs of the animal or animals within that container, or appropriate action can be taken to obtain a homogeneous population. Rearing conditions adapted for a small group of animals based on measurements taken from those animals or their container are more likely to be beneficial to those animals than generic rearing conditions for a large group of animals, which are not based on any measurements. If the measurements and actions are performed on a large group, more animals are likely to be subjected to unsuitable rearing conditions than if those measurements and actions are performed on a small group of animals. This allows the yield of the apparatus to be improved. Isolated containers also allow different species or types of animal to be reared simultaneously using the same apparatus by grouping the animals to different containers.

The measuring device may comprise a thermometer, a humidity sensor or a camera or combinations thereof. The measuring device may be linked with an analyzer such as a computer to analyze the measurement, such as an image analyzer for analyzing images generated by a camera. This is particularly useful when assessing whether a particular animal, for example a mosquito larva, is male or female, or if the animal is healthy. The analyzer may also provide a reliable way of counting the animals in a particular container.

A characteristic of food in the first container which may be measured is whether the food is fit for consumption. This measurement can be used to instruct the actuator to replenish or replace food in the first container. This is useful because food spoilage, and the detrimental impact upon the animal, is a problem with existing animal rearing systems.

The action may be selected from: treating the animal, moving the animal, adding food to the first container or providing an animal with food, removing food from the first container, adjusting the temperature in the first container, adjusting the humidity in the first container, adjusting the pH of contents of the first container, adjusting the illumination of the first container, adding liquid to the first container, removing liquid from the first container, cleaning the first container, adding a cover to or removing a cover from the first container, disposing of an animal in the first container, disposing of contents of the first container, sterilizing an animal, removing contents of the first container and replacing them with fresh contents.

The action may be taking a sample from an animal or the contents of a container. A sample may be taken from the animal for an ex-situ measurement. For example, a cell sample for DNA analysis may be taken. Equally a water sample may be taken from a container for chemical analysis regardless of whether an animal is present in the container.

When the action is the treatment of an animal, the treatment may be surgical or non-surgical and therapeutic or non-therapeutic. Treating the animal may include injecting antibiotics, applying sterilization, adding vitamin or medication supplements to food, or marking the animal's skin with a dye. The action may be replacing contents of the first container with fresh contents.

The action may comprise removing an animal from the first container and depositing the animal in one of a first location and a second location, wherein the actuator is arranged to communicate with the measuring device or the monitoring device and deposit the animal in the first or second location depending on a measurement made by the measuring device or a status monitored by the monitoring device.

The first and second locations may be pots for holding a plurality of animals with similar characteristics or status. Animals may therefore be grouped based on their physical characteristics or status. By grouping of animals with similar characteristics or status, the measurements needing to be performed may be reduced in comparison to individuals or smaller groups. This improves the efficiency of the method. The physical characteristics may be determined by the measurements taken on the animal or container. Grouping the animals by physical characteristics or status is useful for providing homogenous populations, which is commercially desirable. It is also useful for providing control over the genetics of the population as the genetics can be constantly refined by resorting the population. The characteristics or status by which the animals may be sorted include originating batch, measurements during growth and actions taken by the apparatus during growth.

When it is stated herein that components of the apparatus may communicate with one another, it does not mean that those components must communicate directly with one another. For example, the communication may be via an intermediary component such as a computer or other data processor. The intermediary component may add further inputs such as data or calculations which contribute to the determination of the action to be taken by the actuator.

The measurement may be of the characteristics of an animal in the first container or an animal which has been removed from the first container. The actuator may comprise a pipette for removing an animal from the first container. The animal may then be replaced in the same container from which it was removed, or moved to a different container or location.

In some embodiments the actuator comprises a pipette for removing an animal from the first container and the measuring device comprises a camera, wherein the pipette and camera are arranged so that an animal can be removed from the first container with the pipette and photographed.

The animal may then be replaced in the same container from which it was removed, or moved to a different container or location. The actuator may be adapted to perform a manipulation of the first container, the plurality of containers, or an animal removed from the first container, prior to the measurement device taking a measurement.

The manipulation may involve removing an animal from the first container and orientating the animal so that the measurement device can optimize its accuracy, for example to more easily determine the sex of the animal. The manipulation may be in order to enhance the illumination of the first container or an animal in the first container, or an animal removed from the first container. The manipulation may involve removing an animal from the first container with a pipette.

The measurement may be made on the plurality of containers or the environment of the plurality of containers. The actuator may comprise a robotic arm. The apparatus may further comprise a data processing module wherein the data processing module is arranged to communicate with the measurement device or the monitoring device. The data processing module may be arranged to communicate with the actuator. The plurality of containers may be arranged in an array on a tray.

The apparatus may comprise a plurality of trays wherein each tray comprises a plurality of containers. The trays may be arranged in a stack which comprises a plurality of trays. Based on the measurement made by the measurement device, the actuator may be adapted to perform the action on a particular tray or stack. This may involve rearranging the trays in a stack or rearranging the stacks themselves. The containers may be movable or removable individually or in groups or be fixed to the tray (for example, in a setup like a test-tube rack).

The containers may be considered as self-organizing, via programmatic control, with the storage location and/or the processing order for each container being determined dynamically and competitively rather than in fixed batch quantities determined by plate, tray and/or stack organization.

The apparatus may further comprise a timing device which is arranged to communicate with the measuring device or the monitoring device to enable measurements to be taken or statuses to be obtained at specified time intervals. The time intervals may be regular. This may be desirable to allow measurements to be taken more or less frequently dependent on the types of animals being reared or measuring capabilities.

The measuring device may be arranged such that the taking of the measurement is automated. This allows for many measurements and actions to be performed frequently and quickly in comparison to manual operation. This enables the minimum efficient scale of production may be smaller, thereby creating the option to locate production units closer to the point of need. This avoids the need for centralized production and long-distance distribution. This also provides the opportunity for applying machine learning and intelligent software algorithms across multiple production sites, the remote management of production sites and/or license enforcement.

The actuator may be arranged such that an action which it performs is automated. The benefits of automation of the actuator correspond to the benefits of automation of the measuring device as stated above. The measuring device, the monitoring device or the actuator may be adapted to measure, monitor the status of, or perform action on multiple containers simultaneously. The plurality of containers may be wells in a microtiter plate. This type of container is particularly suitable for rearing small insects such as mosquitoes. The action may be providing the animal with nutrients or other treatment by an injection. The use of an injection may be desirable when providing feed or treatment to an animal or animals, to be sure of the exact quantity administered.

The actuator may be arranged to allow the taking of samples from an animal including by phlebotomy. Other sampling techniques forming the state of the art may be utilized by the actuator. Samples may be analyzed by the measuring device after being removed from the animal. In some embodiments the animal may be an aquatic animal.

The height of each container may be greater than both the width and length of each container. Tall and narrow containers are desirable for rearing animals that orient themselves vertically at rest, for example Aedes aegypti larvae. Aquatic animals may also prefer vertical depth for movement. Gravity assisted feeding may be better utilized in this type of container. However, shallow containers may provide more efficient aeration of the water.

The actuator may be arranged to provide an animal or animals with a specific amount of a nutrient. The apparatus may further comprise a computer memory adapted to record measurements taken by the measuring device and/or actions of the actuator, and/or statuses monitored by the monitoring device.

Recorded measurements and/or actions may be used to optimize the rearing process. The recorded measurements and/or actions may be stored in a computer, wherein the computer is able to optimize the rearing process. Parameters that may be optimized may include the type and frequency of measurements, the type and frequency of actions, animals per container, size of container, and conditions of the container at each stage of the rearing cycle, quantity and ingredients of feed at each stage of the rearing cycle.

In a second aspect, the invention provides a method for rearing a plurality of animals comprising the steps of: i) providing animals in a plurality of containers, wherein the number of animals accommodated in each of the plurality of containers is in the range of 1-12, and wherein each container accommodates conditions which are isolated from the conditions in the other containers, ii) performing a measurement on a first container of the plurality of containers, on the environment of the first container, an animal within the first container or an animal removed from the first container, iii) performing an action on the first container, the environment of the first container, an animal in the first container or an animal removed from the first container. The action in step iii) may be determined by the measurement performed in step ii).

The method may comprise the further steps of: iv) repeating step ii) but in relation to a second container of the plurality of containers, the environment of the second container, an animal within the second container or an animal removed from the second container, and v) performing an action on the second container, the environment of the second container, an animal in the second container or an animal removed from the second container, wherein the action taken in step v) may be determined by the measurement taken in step iv). In some embodiments the action taken in step v) is determined by the measurement taken in step iv), and/or the measurement taken in step ii).

Repetition of the measurement and action of the actuator on each isolated container allows small (1-12) groups of animals to have their rearing conditions individually tailored. This provides the advantages discussed above in connection with the first aspect of the invention. In some embodiments there will be 1-8, 1-4 or a single animal in each container. The characteristics of the animal which may be measured are the same as those discussed above in relation to the first aspect of the invention.

The method may further comprise the steps of removing at least one of the animals from a container before performing an action on that container, the environment of that container or the animal removed from that container.

This may be required for particular species of animal or particular actions, such as cleaning the container. The actions may relate to moving an animal to a larger container to allow for further growth of the animal. As animals grow they may require more space within the container to do so, allowing for larger adult animals to be reared.

The method may further comprise the steps of periodically removing all of the animals in a container, cleaning the container and replacing the animals in the container.

The animal(s) may be a mosquito larva or a mosquito and is preferably Aedes aegypti. The animal(s) may be at any stage of their development. For example the animal may be in the form of an egg, a larva, a pupa or an adult. This is particularly useful when the animal is a mosquito, preferably Aedes aegypti. The method may utilize the apparatus described herein.

In some embodiments, in use, water is not recirculated between the plurality of containers. In the case of aquatic animals, this means that solid nutrition can be more readily used and the spread of contamination from dead larvae is prevented.

The sum of the sides (height+width+breadth) of each container may be no greater than 30 cm, or no greater than 20 cm, or no greater than 10 cm. Housing animals in isolated containers wherein the sum of sides is no greater than 30 cm allows small groups of animals to be reared in isolation from other animals.

The volume of each container may be less than 0.0001 m³ or less than 0.00001 m³. The volume of each container may be within the range 1.5×10⁻⁸ m³-3.6×10⁻⁷ m³. The method may comprise steps which correspond to the utilization of any of the features of the apparatus described herein. The apparatus described herein may comprise features which are adapted to provide the method steps described herein.

According to a third aspect of the invention there is provided an apparatus for rearing animals comprising;

a plurality of containers for holding animals, wherein each container is adapted to accommodate controlled conditions which are isolated from the conditions in the other containers, and the volume of each container is no greater than 0.001 m³;

a measuring device which is adapted to perform a measurement on a first container of the plurality of containers or on an animal held within the first container,

wherein the measurement is of; the temperature in the first container, the humidity in the first container, the food quantity within the first container, a characteristic of an animal, the health of an animal, or the gender of an animal in the first container,

the apparatus further comprising an actuator arranged to perform an action on the first container or on an animal in the first container, wherein the action is selected from; moving the animal, providing the animal with a specific amount of a nutrient, adjusting the temperature in the container, adjusting the humidity in the container or disposing of an animal in the container.

The isolation of a particular condition associated with each container, coupled with the measuring device allows the isolated conditions in each container to be monitored accurately. The actuator allows the conditions in a particular container to be changed, or an action to be taken on an animal in a particular container, in response to the specific conditions or the measurement taken on an animal in that particular container. The isolation of the containers, the measuring device and the actuator therefore provide a way of exposing small groups of animals to highly individualized rearing conditions. This can achieve higher yield than could be achieved with growth en masse. If uniform animal fitness is the objective, animals that are measured to be slightly larger than average may require slightly more food while those that are slightly smaller than average may require slightly less food. If the objective is to rear animals of uniform size, actions can be taken to slow the growth of larger individuals and promote the growth of smaller individuals. If the objective is to synchronize growth, for example so that a group of animals enters a life stage such as pupation at a more closely aligned time (which may in turn optimize a storage or transport window), then steps can be taken to slow the development of more developed individuals and accelerate the development of less developed individuals. When the measurement relates to a characteristic of the animal, the characteristic measured may be the animal's size, colour, weight, stage of development, whether the animal is alive, or a combination thereof.

Secondly, reflecting the potential conflict between maximizing animal fitness and achieving uniformity of growth and/or development, the present invention may be used to select, across a much wider range of developing individuals, a subset that meets a specific need. Where animals are reared in a batch process, individuals can be ‘promoted’ or ‘relegated’ between virtual batches to minimize waste and/or maximize quality in the batch. Where animals are reared in a continuous process, the same concept applies by treating each animal as a batch of one.

Thirdly, the ability to vary specific rearing parameters by individual creates significant opportunity to research optimized rearing including (without limitation) feeding and climate.

Fourthly, the measurement may provide the ability to reject an animal as soon as measurements show that it is inappropriate for the objective. This enables feeding and other resources to be stopped as soon as possible, saving costs. Rejected individuals can be removed or sanitized promptly to avoid fungal contamination or other spoiling.

Fifthly, because the measurement may be made on a small number of animals or on an individual animal, quality control is significantly more accurate than in systems based on mass sampling. The actuator may be adapted to add feed or set other conditions based on the exact number of animals rather than the estimated size of a cohort as has been the case in the prior art. The actuator may achieve high accuracy sex separation for ‘sterile insect technique’ applications. The actuator may also be adapted to prevent animals escaping or dying if they are outside the growth norms for a given point in the rearing process (for example, insects becoming adult ‘flyers’).

In some embodiments, in use, water is not recirculated between the plurality containers. In the case of aquatic animals, this means that solid nutrition can be more readily used and the spread of contamination from dead larvae is prevented.

When it is stated that each container is adapted to accommodate controlled conditions which are isolated from the conditions in the other containers, this is intended to cover containers in which only one particular characteristic of the conditions in each container is isolated from the other containers. For example, in some embodiments only the temperature of each container is isolated from the other containers. In other embodiments, only a volume of a growth medium in one container is isolated from a volume of growth medium in other containers. However, two or more of the conditions, for example the temperature and the amount of nutrient in one container may be isolated from the temperature and nutrients in another container.

By regularly measuring the temperature, humidity and food quantity of the container, and the characteristics, health or gender of the animal or animals, the rearing conditions can be adapted to the needs of the animal or animals within that container. Rearing conditions adapted for a small group of animals based on measurements taken from those animals or their container are more likely to be beneficial to those animals than generic rearing conditions for a large group of animals, which are not based on any measurements. If the measurements and actions are performed on a large group, more animals are likely to be subjected to unsuitable rearing conditions than if those measurements and actions are performed on a small group of animals. This allows the yield of the apparatus to be improved. Isolated containers also allow different species or types of animal may be reared simultaneously using the same apparatus by grouping the animals to different containers.

The sum of the sides (height+width+breadth) of each container may be no greater than 30 cm, or no greater than 20 cm, or no greater than 10 cm. Housing animals in isolated containers wherein the sum of sides is no greater than 30 cm allows small groups of animals to be reared in isolation from other animals.

The volume of each container may be less than 0.0001 m³ or less than 0.00001 m³. The volume of each container may be within the range 1.5×10⁻⁸ m³-3.6×10⁻⁷ m³.

The apparatus may be arranged to provide individualised rearing conditions for each individual animal. Individualised rearing allows conditions to be provided that are better suited to each individual animal in order to achieve a higher yield rate. When each container holds only one animal, longer hatching times may be used since the animals will be placed into containers as and when they hatch. This increases the hatching rate and hence the yield.

The measuring device and the actuator may be arranged to communicate so that an action performed by the actuator may be made in response to the results of the measurement performed by the measuring device. The measuring device is able to take measurements to determine the need of the animal or animals in each container. The action can therefore address each of the animal or animals' needs individually, or as a small group, based on the results of that measurement. This provides much more specific rearing conditions than methods of the prior art, which are tailored to individual animals or to small groups of animals. The apparatus may be arranged for rearing mosquitoes.

The apparatus may further comprise trays and stacks, wherein each tray comprises a plurality of containers, each stack comprises a plurality of trays, and the measurement device and the actuator are adapted to perform the actions and measurements on the containers in a tray.

The apparatus may comprise a timing means which is arranged to communicate with the measuring device to enable the measurements to be taken at regular time intervals. This may be desirable to allow measurements to be taken more or less frequently dependent on the types of animals being reared or measuring capabilities.

The measuring device may be arranged such that the taking of the measurement is automated. The actuator may be arranged such that an action which it performs is automated. This allows for many measurements and actions to be performed frequently and quickly in comparison to manual operation. This enables the minimum efficient scale of production may be smaller, thereby creating the option to locate production units closer to the point of need. This avoids the need for centralized production and long-distance distribution. This also provides the opportunity for applying machine learning and intelligent software algorithms across multiple production sites, the remote management of production sites and/or license enforcement.

The apparatus may be arranged such that the measuring device or the actuator is able to measure or perform actions on multiple containers simultaneously, thereby increasing the speed of measurements and actions to be performed.

The containers may be wells in a microtiter plate. This type of container is particularly suitable for rearing small insects such as mosquitoes. The action may be providing the animal with food, in which nutrients are provided by an injection. The use of an injection may be desirable when providing feed to an animal or animals. The actuator may be arranged to allow the taking of samples from an animal by phlebotomy. Other sampling techniques forming the state of the art may be utilised by the actuator. Samples may be analysed by the measuring device after being removed from the animal.

The height of each container may be greater than both the width and length of each container. Tall and narrow containers are desirable for rearing animals that orient themselves vertically at rest, for example Aedes aegypti larvae. Aquatic animals may also prefer vertical depth for movement. Gravity assisted feeding may be better utilised in this type of container. However, shallow containers may provide more efficient aeration of the water.

The actuator may be arranged to provide an animal or animals with a specific amount of a chosen nutrient. Providing nutrients in specific amounts allows for an animal to be reared with desired qualities and improves yield rate.

The measuring device may comprise a thermometer, a humidity sensor or a camera. When the measuring device is a camera, it may be linked with an analyser such as a computer to analyse the images generated by the camera. This is particularly useful when assessing whether a particular animal, for example a mosquito larvae is male or female, or if the animal is healthy. The analyser may also provide a reliable way of counting the animals in a particular container.

In preferred embodiments the measuring devices and actuators are connected to a control system, such as a computer. This allows data from the measuring devices and actuators to be recorded and reviewed systematically. It also allows the control system to use data from the measuring devices to determine whether an actuator should carry out a specific action on a particular container.

According to a fourth aspect of the invention there is provided a method for rearing a plurality of animals comprising the steps of;

i) providing animals in a plurality of containers, wherein the number of animals accommodated in each of the plurality of containers is in the range of 1-12, and wherein each container accommodates controlled conditions which are isolated from the conditions in the other containers,

ii) performing a measurement on a first container of the plurality of containers or on an animal held within the first container, wherein the measurement is of the temperature in the first container, the humidity in the first container, the food quantity in the first container, a characteristic of the animal, the health of the animal or the gender of the animal;

iii) performing an action on the first container or on the animals accommodated in the first container in response to the measurement taken in step ii), wherein the action is moving the animal, providing the animal with food, adjusting the temperature or humidity in the container or disposing of the animal, and

iv) repeating step ii) but in relation to a second container or on an animal in a second container and subsequently repeating step iii) in response to the measurement taken in connection with the second container or on the animal in the second container.

Repetition of the measuring and action of the actuator on each isolated container allows small (1-12) groups of animals to have their rearing conditions individually tailored. This provides the advantages discussed above in connection with the third aspect of the invention. In some embodiments there will be 1-8, 1-4 or a single animal in each container. The characteristics of the animal which may be measured are the same as those discussed above in relation to the third aspect of the invention.

The method may further comprise the steps of removing at least one of the animals from a container before performing an action on that container or animal. This may be required for particular species of animal or particular actions, such as cleaning the container. The method may further comprising the steps of periodically removing all of the animals in a container, cleaning the container and replacing the animals in the container.

The actions may relate to moving an animal to a larger container to allow for further growth of the animal. As animals grow they may require more space within the container to do so, allowing for larger adult animals to be reared. The actions may relate to grouping of animals based on their physical characteristics. By grouping of animals with similar characteristics, the measurements and needing to be performed may be reduced in comparison to individuals or smaller groups. This improves the efficiency of the method. The physical characteristics may be determined by the measurements taken on the animal or container.

The measurements and actions may be recorded in a memory. Recorded measurements and actions may be used to optimize the rearing process. The recorded measurements and actions may be stored in a computer, wherein the computer is able to optimize the rearing process. Parameters that may be optimized may include frequency of measurements, frequency of actions, animals per container and size of container. The method may utilize the apparatus described herein. Preferably the animal(s) is a mosquito larvae or a mosquito and is preferably Aedes aegypti. In some embodiments the animal may be an aquatic animal.

### Further Statements of Disclosed Subject Matter

In accordance with an aspect of the present disclosure, an apparatus for rearing animals is provided which comprises:
a plurality of containers for holding animals, wherein each container is adapted to accommodate controlled conditions which are isolated from the conditions in the other containers, and the volume of each container is no greater than 0.001 m³;
a measuring device which is adapted to perform a measurement on a first container of the plurality of containers or on an animal held within the first container,
wherein the measurement is of;
the temperature in the first container, the humidity in the first container, the food quantity within the first container, a characteristic of an animal, the health of an animal, or the gender of an animal in the first container,
the apparatus further comprising an actuator arranged to perform an action on the first container or on an animal in the first container,
wherein the action is selected from;
moving the animal, providing the animal with food, adjusting the temperature in the container, adjusting the humidity in the container or disposing of an animal in the container.

In some embodiments, the measuring device and the actuator are arranged to communicate so that an action performed by the actuator may be made in response to the results of the measurement performed by the measuring device.

In some embodiments, the apparatus further comprises trays and stacks, wherein each tray comprises a plurality of containers, each stack comprises a plurality of trays, and the measurement device and the actuator are adapted to perform the actions and measurements on the containers in a tray.

In some embodiments, the apparatus further comprises a timing means which is arranged to communicate with the measuring device to enable the measurements to be taken at regular time intervals. In some embodiments, the measuring device is arranged such that the taking of the measurement is automated. In some embodiments, the actuator is arranged such that an action which it performs is automated. In some embodiments, the apparatus is arranged such that the measuring device or the actuator is able to measure or perform actions on multiple containers simultaneously. In some embodiments, the containers are wells in a microtiter plate. In some embodiments, the action is providing the animal with food, in which nutrients are provided by an injection. In some embodiments, the height of each container is greater than both the width and length of each container. In some embodiments, the actuator is arranged to provide an animal or animals with a specific amount of a chosen nutrient.

In accordance with another aspect of the disclosure, a method for rearing a plurality of animals is provided which comprises the steps of:
i) providing animals in a plurality of containers, wherein the number of animals accommodated in each of the plurality of containers is in the range of 1-12, and wherein each container accommodates conditions which are isolated from the conditions in the other containers,
ii) performing a measurement on a first container of the plurality of containers or on an animal held within the first container,
wherein the measurement is of the temperature in the first container, the humidity in the first container, the food quantity in the first container, a characteristic of the animal, the health of the animal or the gender of the animal;
iii) performing an action on the first container or on the animal accommodated in the first container in response to the measurement taken in step ii),
wherein the action is moving the animal, providing the animal with food, adjusting the temperature or humidity in the container or disposing of the animal, and
iv) repeating step ii) but in relation to a second container or on an animal in a second container and subsequently repeating step iii) in response to the measurement taken in connection with the second container or on the animal in the second container.

In some embodiments, the method further comprises removing at least one of the animals from a container before performing an action on that container or animal. In some embodiments, the method further comprises periodically removing all of the animals in a container, cleaning the container and replacing the animals in the container. In some embodiments, the actions relate to moving an animal to a larger container to allow for further growth of the animal. In some embodiments, the actions relate to grouping of animals based on their physical characteristics. In some embodiments, the measurements and actions are recorded in a memory. In some embodiments, the method utilizes the apparatus described herein. In some embodiments, the animal(s) is a mosquito larvae or a mosquito and is preferably Aedes aegypti.

A feature or features of any aspect of the invention described herein may be combined in some embodiments with any feature or features of any other aspect of the invention described herein.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 shows the process flow for a rearing method according to the present invention.

FIG. 2 shows a schematic view of the apparatus used in the method of FIG. 1.

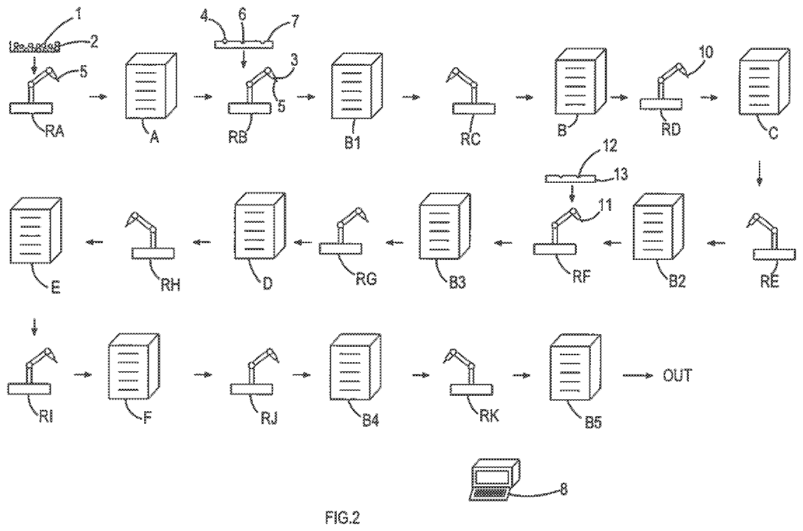

### DETAILED DESCRIPTION OF AN EXEMPLARY EMBODIMENT

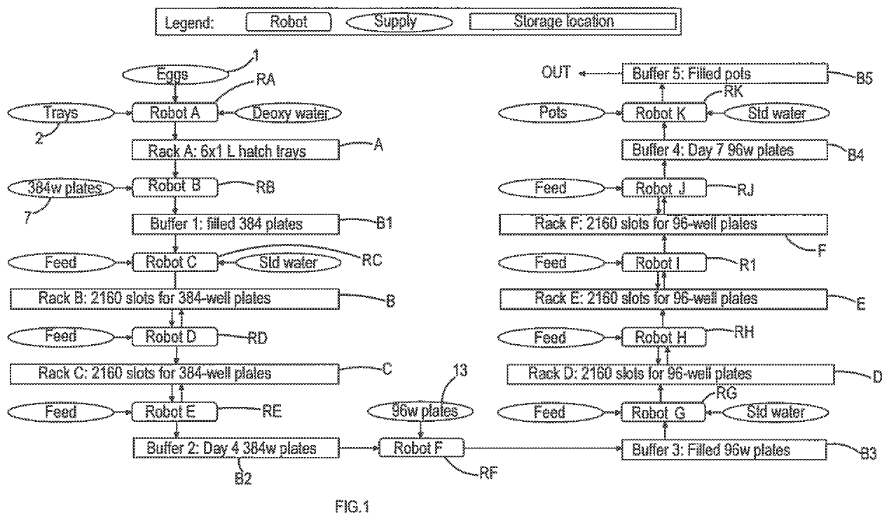

The process of FIG. 1 is arranged for the rearing of Aedes aegypti mosquitoes. The process is automated using robots RA-RK. The process begins by taking Aedes aegypti eggs 1. Aedes aegypti eggs are prone to clumping, hence the eggs 1 are measured by weight into batches of about 18,000 for hatching. The weighing is calibrated at intervals and adapted to achieve the correct number of eggs 1 for hatching.

Hatching trays 2 are reused once the eggs 1 have hatched. Robot RA periodically removes the oldest used hatching tray 2 from rack A. Robot RA pours the tray contents through a fine mesh filter so that captured solids are able to be disposed of, and liquids are able to be recycled. Robot RA then cleans the tray 2 and places a batch of 18,000 eggs 1 and 1 litre of deoxygenated water into the tray 2 and returns the tray 2 to rack A. The tray 2 is now in-use.

Robot RB periodically retrieves each of the in-use trays 2 from rack A. Robot RB utilises a camera 3 to identify hatched larvae 4 and unhatched eggs 1. The diameter of the larvae 4 are also measured using the camera. Aedes aegypti larvae are transparent and roughly spherical with a diameter of about 1 mm, this is in contrast to Aedes aegypti eggs which are black. Each hatched larva 4 identified by robot RB is removed by robot RB using a first pipette 5 and placed in a vacant well 6 in a 384-well microtiter plate 7. Simultaneously the well number, the time the larva 4 was removed from the tray 2, how long that larva 4 had been in the tray 2 and the diameter of the larvae 4 are recorded by an information system 8. A hatching time limit of 6 hours is used.

When every well 6 of the plate 7 has been filled, robot RB moves the plate to a first buffer B1 comprising a two-sided rack operating a first-in-first-out system.

Robot RC periodically retrieves the oldest plate from the first buffer B1 and uses water to top up the level in each well 6 to a total of 90 microliters, including the larva 4 and any material transferred with it by robot RB. Robot RC then adds a measured standard quantity of nutrition in a liquid form to each well 6 and returns the plate to the next vacant slot of rack B. Each plate 7 is allowed 48 hours growing time in rack B.

On the other side of rack B, robot RD periodically removes a plate 7 for inspection. Robot RD utilises optical sensors 10 to calculate the size of the larvae 4. If a larva 4 is below a minimum size threshold for its growing time, nutrition is added. The quantity and ingredients of the nutrition is calculated according to the measurements of the larva 4 and its previous feeding history. If a larva 4 is dead, it will be disposed of or marked virtually as disposed of. The measurements and actions performed on each larva 4 are recorded using the information system 8, which is connected, wirelessly or otherwise (not shown), to each of the sensors in the system. The plate 7 is then returned to rack B.

If the death rate on any one plate 7 exceeds a certain value, or the aggregate death rate across a set of plates 7 at a similar stage exceeds a certain lower value, an alarm is given to a human operator to decide if manual intervention is needed.

Once a plate 7 has been in rack B for 48 hours, robot RD places the plate 7 in a two-sided rack C operating a first-in-first-out system. On the other side of rack C, robot RE periodically removes a plate 7 for inspection. Robot RE operates in an identical way and follows the steps of robot RD described above. Once a plate 7 has been in rack C for 48 hours, robot RE places the plate 7 in second buffer B2, which comprises a two-sided rack operating a first-in-first-out system.

On the other side of the second buffer B2, robot RF periodically removes the oldest plate 7 from the second buffer B2. Robot RF determines the probability that each larva is a female using a visual recognition system. Robot RF then uses a second pipette 11 to remove the larvae 4 from the plate 7 and place each larva 4 into an empty well 12 in a 96-well microtiter plate 13. A larva will be rejected only if there is a very high probability, over 98%, that it is a female. A larva identified as a female is moved to a system for rearing females.

Once a 384-well plate 7 has been processed, the contents are emptied to remove solid matter and the remaining liquid is treated for recycling. The plate 7 is then cleaned and reused. Robot RF places each 96-well plate 13 in a third buffer B3 comprising a two-sided rack operating a first-in-first-out system.

On the other side of the third buffer B3, a robot RG periodically takes the oldest plate from buffer B3. Robot RG tops up the water in each well 12 to a total of 235 microliters, including the larva and any material transferred with it. Robot RG then adds a measured standard quantity of nutrition in a liquid form to each well 12 and returns the plate 13 to the next vacant slot of a two-sided rack D.

Robot RH periodically retrieves the plates 13 from rack D for inspection. The larvae are allowed 24 hours of growing time in rack D. The inspection process performed by robot RH is identical to that performed by robots RD and RE described above. The measurements and actions performed on each larva 4 are recorded.

Once a plate 13 has been in rack D for 24 hours, robot RH places the plate 13 in a two-sided rack E. The process is repeated for racks E and F as for rack D. The plates are placed in rack F by a robot RI after growing in rack E.

Once a plate 13 has been in rack F for 24 hours, robot RJ places the plate 13 in a fourth buffer B4 comprising a two-sided rack operating a first-in-first-out system. As the fourth buffer B4 contains larvae 4 that have been growing for seven days, since being first placed into rack B, some of the larvae 4 will have turned into pupae.

On the other side of the fourth buffer B4, a robot RK retrieves the oldest plate from the fourth buffer B4 and determines the probability that each larva/pupa 4 is female. Robot RK will discard a larva/pupa 4 if there is even a small possibility, over 5%, that it is female. Robot RK then uses a pipette to remove the larva or pupa 4, that have not been virtually marked as discarded, to a pot. Once each pot contains 300 larva/pupa 4, robot RK adds a meshed lid to prevent escape of the larva/pupa 4 and places the pots in a fifth buffer B5, from which the pots are taken for distribution.

Once all of the wells 12 in a 96-well plate 13 are emptied, the solid matter is disposed of and the remaining liquid is treated for recycling. The plate 13 is then cleaned and reused.

1. An apparatus for rearing animals comprising;
a plurality of containers for holding animals, wherein each container is adapted to accommodate controlled conditions which are isolated from the conditions in the other containers, and the volume of each container is no greater than 0.001 m³,
a measuring device which is adapted to perform a measurement on a first container of the plurality of containers, the environment of the first container, an animal within the first container or an animal removed from the first container, and
an actuator arranged to perform an action on the first container, the environment of the first container, an animal in the first container or an animal removed from the first container.

2. An apparatus according to claim 1 further comprising a monitoring device for monitoring a status, wherein the status is of the apparatus, an animal associated with the apparatus or a process associated with the apparatus.

3. An apparatus according to claim 1, wherein the measuring device is arranged to communicate with the actuator so that an action performed by the actuator may be made in response to a measurement obtained by the measuring device.

4. An apparatus according to claim 1 wherein the measurement is of;
the temperature in the first container, the humidity in the first container, the illumination of the first container, the turbidity of contents of the first container, the pH of contents of the first container, food quantity within the first container, a characteristic of food in the first container, a characteristic of an animal such as the dimensions of an animal, weight of an animal, colour of an animal, health of an animal, or the sex of an animal in the first container, the cleanliness of the first container, or a combination thereof

5. An apparatus according to claim 1 wherein the action is selected from;
treating the animal, moving the animal, adding food to the first container or providing an animal with food, removing food from the first container, adjusting the temperature in the first container, adjusting the humidity in the first container, adjusting the pH of contents of the first container, adjusting the illumination of the first container, adding liquid to the first container, removing liquid from the first container, cleaning the first container, adding a cover to or removing a cover from the first container, disposing of an animal in the first container, disposing of contents of the first container, sterilizing an animal, removing contents of the first container and replacing them with fresh contents.

6. An apparatus according to claim 1 wherein the action comprises removing an animal from the first container and depositing the animal in one of a first location and a second location, wherein the actuator is arranged to communicate with the measuring device and deposit the animal in the first or second location depending on a measurement made by the measuring device.

7. An apparatus according to claim 1 wherein the actuator comprises a pipette for removing an animal from the first container.

8. An apparatus according to claim 1 wherein the actuator comprises a pipette for removing an animal from the first container and the measuring device comprises a camera, wherein the pipette and camera are arranged so that an animal can be removed from the first container with the pipette and photographed.

9. An apparatus according to claim 1 wherein the actuator is adapted to perform a manipulation of the first container, the plurality of containers, or an animal removed from the first container, prior to the measurement device taking a measurement.

10. An apparatus according to claim 1 wherein the measurement is made on the plurality of containers or the environment of the plurality of containers.

11. An apparatus according to claim 1 wherein the actuator comprises a robotic arm.

12. An apparatus according to claim 1 further comprising a data processing module wherein the data processing module is arranged to communicate with the measurement device.

13. An apparatus according to claim 1 wherein the plurality of containers are arranged in an array on a tray.

14. An apparatus according to claim 1, further comprising a timing device which is arranged to communicate with the measuring device to enable measurements to be taken at specified time intervals.

15. An apparatus according to claim 1, arranged such that the measuring device or the actuator is adapted to measure or perform action on multiple containers simultaneously.

16. An apparatus according to claim 1 wherein the action is providing the animal with nutrients or other treatment by an injection.

17. An apparatus according to claim 1, wherein the height of each container is greater than both the width and length of each container.

18. An apparatus according to claim 1, wherein the actuator is arranged to provide an animal or animals with a specific amount of a nutrient.

19. An apparatus according to claim 1, further comprising a computer memory adapted to record measurements taken by the measuring device and/or actions of the actuator.

20. A method for rearing a plurality of animals comprising the steps of;
i) providing animals in a plurality of containers, wherein the number of animals accommodated in each of the plurality of containers is in the range of 1-12, and wherein each container accommodates conditions which are isolated from the conditions in the other containers,
ii) performing a measurement on a first container of the plurality of containers, on the environment of the first container, an animal within the first container or an animal removed from the first container,
iii) performing an action on the first container, the environment of the first container, an animal in the first container or an animal removed from the first container.
